(19) United States
(12) Plant Patent Application Publication
(10) Pub. No.: US 2003/0051276 P1
(43) Pub. Date: Mar. 13, 2003

(54) Interspecific Dianthus plant

(76) Inventors: Kerry Strope, Pismo Beach, CA (US); Scott Trees, Shell Beach, CA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 09/817,823

(22) Filed: Mar. 26, 2001

Related U.S. Application Data
(60) Provisional application No. 60/192,261, filed on Mar. 27, 2000.

(51) Int. Cl. A01H 1/00; A01H 1/02; A01H 5/00; C12N 5/00; C12N 5/02

(52) U.S. Cl. 800/323.3; 800/323; 800/270; 435/410

(57) ABSTRACT

The present invention relates to a novel interspecific Dianthus plant. The Dianthus plant of the present invention was developed through a unique interspecific cross between Dianthus caespitosus and Dianthus barbatus.

### RELATED APPLICATION INFORMATION

This application claims priority from U.S. Application No. 60/192,261 filed on Mar. 27, 2000.

### FIELD OF INVENTION

The present invention relates to a novel interspecific Dianthus plant. The interspecific Dianthus plant of the present invention was developed through a unique interspecific cross between Dianthus caespitosus and Dianthus barbatus.

This invention also relates to interspecific Dianthus seed, interspecific Dianthus plants, interspecific Dianthus varieties, interspecific Dianthus hybrids and interspecific Dianthus flower mutants.

In addition, the present invention also relates to methods for transferring heat tolerance to Dianthus varieties using Dianthus caespitosus in breeding as either a female or male parent, in order to produce novel types and varieties of interspecific Dianthus plants which exhibit a heat tolerance trait. The present invention also relates to a F₁ hybrid or later generation interspecific Dianthus plant grown from the interspecific hybrid seed produced by the aforementioned methods.

### BACKGROUND OF INVENTION

The genus Dianthus, commonly known as pinks, consists of more than 300 annual, biennial and perennial herbaceous species originating mainly from Europe, Asia and extending south to Africa. The showy, often fragrant flowers are mostly pink to rose, but include red, purple, white and yellow. In the ornamental industry, Dianthus cultivars are used as cut flowers, pot plants and bedding plants. Bedding plant cultivars are well suited to rock gardens since many species come from alpine habitats. (Hortus Third A Concise Dictionary of Plants Cultivated in the United States and Canada, MacMillan Publishing Company (1976)).

Interspecific hybridization has been used in Dianthus cultivar improvement. In particular, crosses between Dianthus chinensis, the early and long-flowering annual with a compact habit, and Dianthus barbatus, the cold hardy Sweet William with a broad color range, have yielded improved hybrids for fall planting (A. Armitage, Greenhouse Grower, June: 75-78 (1999)). To evaluate for early flowering and response to day length, interspecific crosses involving ten Dianthus species (D. caryophyllus, D. chinesis, D. superbus, D. barbatus, D. japonicus, D. brachyzonus, D. versicolor, D. trifasciculatus, D. serotinus and D. knappii) were generated. Progenies of D. japonicus and D. chinensis demonstrated the ability to flower early and abundantly under low light intensity and a short light period (L. D. Sparnaaij and H. J. J. Koehorst-van Putten, Euphytica 50: 211-220, (1990)). Embryo rescue techniques were used to recover interspecific hybrids between the female parent Dianthus caryophyllus and the male parents D. chinensis and D. barbatus for use in breeding for increased tolerance to extreme temperatures and increased disease resistance. In this report, a variable population of interspecific hybrids were produced; however, no tolerance to extreme temperatures or disease resistance was identified (G. B. Wen, Acta Horticulturae 404:82-90, (1993)). In addition, interspecific hybridization has been carried out with the aim of transferring yellow flower color into varieties having bedding plant habits. Pale cream-yellow hybrids were produced from crossing a white flowered Dianthus plumarius with yellow flowered wild species, Dianthus knappii (M. K. Gatt, Scientia Horticulturae 77: 207-218, (1998)).

To modify flower color pattern, variants of Dianthus caryophyllus, carnation, have been produced through mutagenic treatment. Flower pattern variation, i.e. dots, streaks or basic color changes, were recovered through organogenesis of gamma irradiated in vitro petal cultures (M. Simard, N. Michaux-Ferriere and A. Silvy, Plant Cell, Tissue and Organ Culture 29:37-42, (1992)).

### SUMMARY OF INVENTION

The present invention relates to novel interspecific Dianthus plants produced as a result of a unique cross which has previously never before been attempted. More specifically, the present invention relates to interspecific Dianthus plants produced from a cross between a Dianthus caespitosus and Dianthus barbatus. The Dianthus caespitosus and Dianthus barbatus plants can each be used as either the male or female parent. Interspecific Dianthus plants produced as a result of this cross are heat tolerant. The present invention also relates to seed, pollen, cuttings and ovules of an interspecific Dianthus plant produced from this cross as well as to a tissue culture comprising regenerable cells of an interspecific Dianthus plant produced from this cross.

The present invention also relates to interspecific Dianthus plants having a novel heat tolerance trait. The interspecific Dianthus plants of the present invention possesses heat tolerance and have a pedigree which includes BFP-2081 or derivatives thereof. The present invention also relates to seed, pollen, cuttings and ovules of the heat tolerant interspecific Dianthus plants of the present invention. Moreover, the present invention also relates to a tissue culture comprising regenerable cells of the heat tolerant interspecific Dianthus plants of the present invention.

Additionally, the present invention relates to interspecific Dianthus seeds which contain the heat tolerance trait. The seed of the present invention has a pedigree which includes BFP-2081. The present invention also relates to a heat tolerant interspecific Dianthus plant produced by growing the seed of the present invention.

The present invention also relates to a Dianthus plant having a lineage which includes the interspecific Dianthus plant BFP-2081 and which exhibits at least one of the following characteristics: heat tolerance, vigorous, well-branched, trailing habit, five-petaled pink flowers, free-flowering, continuous blooming and grass-like foliage.

The present invention also relates to an interspecific Dianthus plant that has been subjected to a mutation treatment resulting in plants exhibiting altered flower color or color pattern. The treatment involves irradiating an interspecific Dianthus plant with electromagnetic radiation, ion beams and then selecting a shoot from the treated plant containing a flower having at least one petal which exhibits an altered flower color or color pattern. The irradiated plant may be a rooted cutting or a whole plant.

The present invention also relates to a method for transferring heat tolerance from Dianthus caespitosus into other Dianthus varieties, such as, but not limited to, Dianthus barbatus. The method involves crossing pollen from a first parent Dianthus plant to a second parent Dianthus plant and harvesting the resultant first generation (F₁) hybrid Dianthus seed. One of the parent Dianthus plants used in said method must be a Dianthus caespitosus. Additionally, the present invention relates to a first generation (F₁) hybrid plant produced by growing the hybrid seed produced by said method.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 shows a photograph of a Dianthus caespitosus×Dianthus barbatus hybrid named BFP-2081 of the present invention in a garden location.

FIG. 2 shows a photograph of altered flower color and color patterns of a Dianthus caespitosus×Dianthus barbatus hybrid named BFP-2081 of the present invention.

### DETAILED DESCRIPTION OF THE INVENTION

The present invention relates to interspecific Dianthus plants developed through a unique interspecific cross between Dianthus caespitosus and Dianthus barbatus, which has never before been attempted. The interspecific Dianthus plants produced as a result of this cross exhibits the trait of heat tolerance.

As used herein, the term “heat tolerance” or “heat tolerant” is defined as a plant able to withstand average daily temperature above 86° F. (30° C.) for at least 4 weeks length of time while continuing to flower.

As used herein, the term “altered flower color” means the color on a flower petal of a plant has been changed, such as, but not limited to, being lightened or darkened, when compared to a control plant not treated pursuant to the method hereinafter described.

As used herein, the term “altered color pattern” means that a darker background color on a flower petal is randomly interdispersed with irregular bands or streaks of lighter color.

This previously unknown interspecific Dianthus plant was discovered as a result of breeding and research efforts which were conducted at Arroyo Grande, Calif. In 1997, a cross was made using Dianthus caespitosus as the female parent (purchased from Silverhill Seeds, P.O. Box 53108, Kenilworth, 7745 Cape Town, South Africa). Dianthus caespitosus is native to South Africa. The Dianthus caespitosus used in the cross described herein exhibits pale pink to light yellow flowers with non-overlapping petals. It has grass-like foliage of medium green color. The habit is mounded trailing. The male parent was Dianthus barbatus “Wee Willie” Mix (commercially available from Ball Seed Company, 622 Town Road, West Chicago, Ill. 60185). Dianthus barbatus is native to the Pyrenees, Carpathian Mountains and Balkan Pennisula. The Dianthus barbatus used in the cross described herein is an open-pollinated Sweet William type early flowering variety with single medium-pink blooms on well-branched 3 to 6-inch plants. In 1997, the resulting F₁ seed was collected and germinated. From the flowering progeny, a plant identified as BFP-2081 was selected.

The interspecific Dianthus plant BFP-2081 possesses a number of unique characteristics. Specifically, heat tolerance, vigorous, well-branched, trailing habit, five-petaled pink flowers, free-flowering, continuous blooming and grass-like foliage.

BFP-2081 has not been observed under all possible environmental conditions. The phenotype may vary significantly with variations such as temperature, light intensity and daylength, without, however, any variance in genotype.

Additionally, the interspecific Dianthus plant BFP-2081 of the present invention has been subjected to a mutation treatment resulting in plants exhibiting altered flower color or color patterns. These interspecific Dianthus plants having altered flower color or color patterns arose as a result of the irradiation of the interspecific Dianthus plant BFP-2081 of the present invention with electromagnetic radiation, particularly, gamma rays. It is believed that the irradiation of an interspecific Dianthus plant causes a somatic mutation in the cells which produce the petals and thus results in a chimera having flowers with altered flower color or color patterns.

It is believed that the altered flower color and color patterns in the petals of an interspecific Dianthus plant can also be obtained in an interspecific Dianthus by irradiating a plant with other types of electromagnetic radiation such a x-rays and ultraviolet rays as well as ion beams. The interspecific Dianthus plant may be irradiated with from about 1.5 to about 3.5 kilorads of electromagnetic radiation or ion beams. Preferably, the interspecific Dianthus plants are irradiated with from 2.0 to 3.0 kilorads of radiation or ion beams.

A cutting rooted in a suitable growth medium, such as oasis, or an entire interspecific Dianthus plant may be irradiated using the processes described herein. If a rooted cutting is used, once irradiated, the cutting is allowed to grow. Shoots from the treated rooted cutting containing flowers having petals exhibiting altered flower color or color patterns described herein are then selected and propagated through cuttings or other forms of asexual reproduction. If an entire interspecific Dianthus plant is irradiated, shoots from the treated plant containing flowers having petals exhibiting altered flower color or color patterns are selected and propagated through asexual propagation.

The heat tolerant interspecific Dianthus plants of the present invention are genetically stable and can be stably reproduced by means of asexual propagation. Cuttings for asexual propagation can be taken at any time of the year and no special hormones or soil mixtures are required. It is expected that any heat tolerant interspecific Dianthus can be produced commercially through asexual propagation.

While the interspecific Dianthus plants of the present invention are not sterile they maintain very low fertility and can thus be employed as a female and/or male parent in traditional breeding. Methods for overcoming interspecific hybrid sterility barriers are known in the art and include, but are not limited to, embryo rescue, colchicine treatments, random assertive mating and naturally developing pollen fertility.

Using the methods described herein, it is expected that the heat tolerance trait from Dianthus caespitosus can be bred into diverse Dianthus backgrounds, including those having white, yellow, pink, rose, red or purple flower color, as well as bicolor flowers. Flower forms can include single, double, over-lapping petals, non-overlapping petals, petals with fringed edges, ruffled appearance or exaggerated fringes. Additionally, the heat tolerance trait can be incorporated into Dianthus having foliage colors ranging from light to dark green or blue-green and foliage types ranging from grass-like or strap-like to sword-like.

The following examples are set forth as representations of specific and preferred embodiments of the present invention. These examples are not to be construed as limiting the scope of the invention in any manner. It should be understood that many variations and modifications can be made while remaining within the spirit and scope of the invention.

### EXAMPLE 1

### Detailed Description of Dianthus caespitosus×Dianthus barbatus Hybrid Named BFP-2081 and Comparison with Dianthus deltoides ‘Zing Rose’

The color chart used in the identification of colors described herein is the R.H.S. Colour Chart of The Royal Horticultural Society, London, England. The color values were determined on Mar. 13, 2000 in West Chicago, Ill. The readings were taken between 1:00 p.m. and 1:45 p.m. under approximately 2500 footcandles of light.

The plants were produced from cuttings taken from stock plants and were grown under greenhouse conditions comparable to those used in commercial practice while utilizing a soiless growth medium and maintaining temperatures of approximately 72° F. during the day and approximately 65° F. during the night. Dianthus deltoides ‘Zing Rose’ is commercially available from White Flower Farm, P.O. Box 50, Litchfield, Conn. 06759.

| CHARACTERISTIC | BFP 2081 | Zing Rose |
|---|---|---|
| Plant form | Trailing | Upright to trailing |
| Growth habit | Vigorous | Medium |
| Plant Height | 16 cm | 22 cm |
| Lateral branch diameter | 2 mm | 1 mm |
| Internode length | 4.2 cm | 2.9 cm |
| Stem color | 137C | 137A |
| Stem texture | Smooth | Same |
| Leaf arrangement | Opposite | Same |
| Leaf length | 5.3 cm | 3.5 cm |
| Leaf width | 4 mm | 5 mm |
| Leaf shape | Linear | Same |
| Leaf apex | Acute | Same |
| Leaf base | United forming sheath | Same |
| | around stem | |
| Leaf margin | Entire | Same |
| Leaf texture | Smooth | Same |
| Leaf aspect | Flat | Same |
| Leaf color-upper surface | 137A | 137A |
| Leaf color-lower surface | 137B | 137A |
| Flower shape | Round | Same |
| Flower Type | Explanate | Same |
| Flower arrangement | Paniculate | Same |
| Flower bud length | 2 cm | 2.5 cm |
| Flower bud shape | Cylindrical | Same |
| Flower bud diameter | 3.3 mm | 4 mm |
| Petal arrangement | Nonimbricate | Same |
| Petal shape | Broad limb attenuated | Same |
| | into elongated claw | |
| Petal quantity | 5 | Same |
| Petal: limb length | 1.3 cm | 1.7 cm |
| Petal: limb width | .8 cm | 1.4 cm |
| Petal: claw length | 1.9 cm | 2.2 cm |
| Petal claw width | 1 mm | 2 mm |
| Petal apex | Rounded | Flat to slightly |
| | | rounded |
| Petal margin | Edges entire; tip | Same |
| | fringed | |
| Petal texture | Smooth | Smooth with hairs at |
| | | throat |
| Petal color: upper surface | Closest to 72C | Brighter than 61B |
| Petal color: lower surface | 73C | 64A |
| Sepal shape | Linear | Same |
| Sepal length | 1.9 cm | 2.2 cm |
| Sepal width | 2 mm | 3 mm |
| Sepal quantity | 5 | Same |
| Calyx length | 1.9 cm | 2.2 cm |
| Calyx type | Tubular | Same |
| Calyx diameter | 4 mm | 5 mm |
| Calyx apex | 5 toothed | Same |
| Calyx texture | Smooth | Same |
| Calyx color | Between 137C and | Between 137A and |
| | 137D | 137B |
| Epicalyx | 2 pairs of scales | 1 pair of scales |
| Stamen number | 10 | Same |
| Stamen length | 1.3 cm | 2.2 cm |
| Stamen color | White | Same |
| Anther color | Greyish Brown | Bluish Purple |
| Filament length | 1.2 cm | 2.1 cm |
| Pistil length | 3.1 cm | 2.1 cm |
| Ovary length | 9 mm | 9 mm |
| Ovary color | Closest to 145A | Same |
| Stigma lobes | 2 | 3 |
| Stigma shape | Linear | Same |
| Stigma length | 13 mm | 8 mm |
| Stigma color | White with white | White with purple |
| | fringe, curled at tip | fringe |
| Style length | 9 mm | 4 mm |
| Style color | White | Same |
| Filament color | White | Same |

### EXAMPLE 2

### Evaluation of Heat Tolerance of Dianthus caespitosus×Dianthus barbatus Hybrid Named BFP-2081

In 1999, the interspecific Dianthus plant BFP-2081 of the present invention was evaluated in a field trial conducted in West Chicago, Ill. Plants were grown throughout the summer in 5-gallon containers exposed to full sunlight. Grown under these conditions, BFP-2081 continued to set bloom and flower prolifically with only slight color fading and without any reduction in flower size. In addition there was no appearance of leaf burn. A comparison cultivar of the Telstar series (available from Ball Seed Company, 622 Town Road, West Chicago, Ill. 60185) had all but one out of three plants die and that remaining plant exhibited leaf burn. The Telstar series is composed of Dianthus barbatus and Dianthus chinensis hybrids. It should be noted that Dianthus barbatus is the male parent of plant BFP-2081.

### EXAMPLE 3

### Irradiation Program to Develop Flower Pattern and Color Mutants of Dianthus caespitosus×Dianthus barbatus Hybrid Named BFP-2081

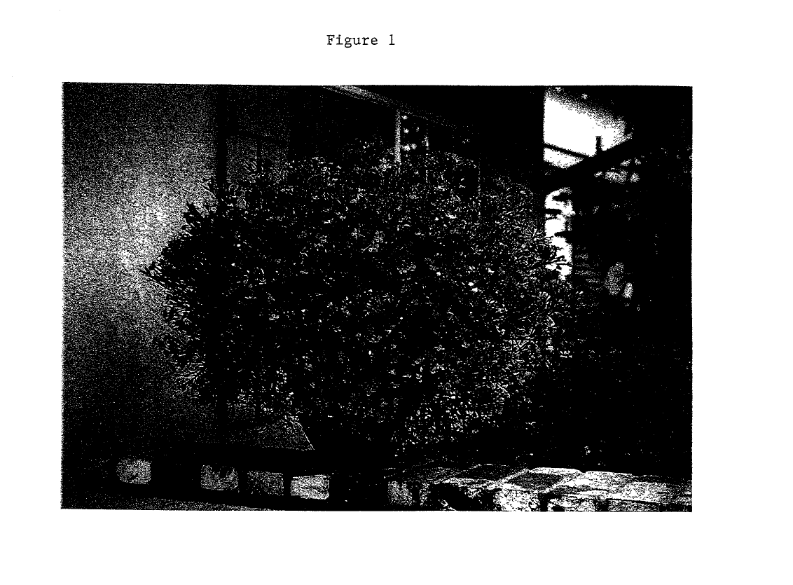

The interspecific Dianthus plant BFP-2081 of the present invention is heat tolerant, vigorous, well-branched, has a trailing habit, has five-petaled medium pink flowers, is free-flowering, is continuous blooming and has grass-like foliage. The petals are closest to 72° C. upper surface and 73° C. lower surface. As shown in FIG. 1, the petals of BFP-2081 do not exhibit flower color patterns.

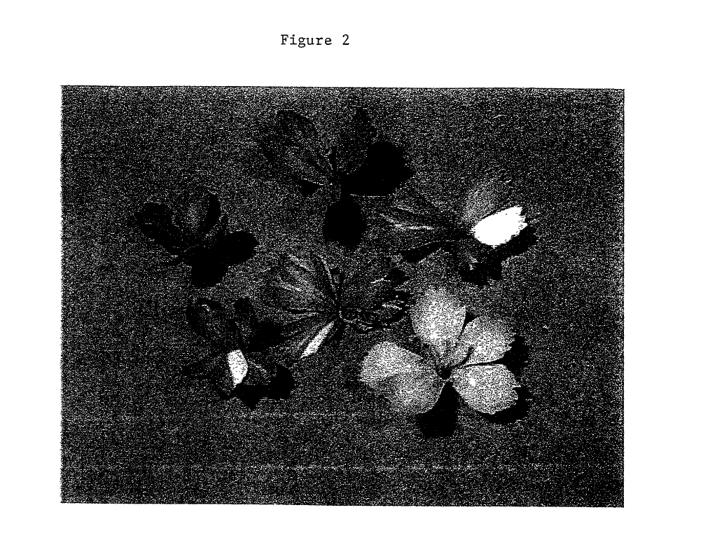

A mature plant from BFP-2081 was irradiated with 2 kilorads of gamma rays. The irradiated plant was allowed to grow. Cuttings having flowers containing petals which exhibited altered flower color or color patterns were made and allowed to grow. The resulting plants contained a number of flowers having petals which exhibited the altered flower color or color patterns. Cuttings having these flowers containing the petals with the altered flower color or color patterns were made and planted. As shown in FIG. 2, the petals can display a range of altered flower color patterns.

Deposit Information

Two thousand five hundred (2500) seeds of Dianthus caespitosus have been placed on deposit with the American Type Culture Collection (ATCC), 10801 University Blvd., Manassas, Va., 20110-2209 under Deposit Accession Number PTA-1531 on Mar. 21, 2000. These seeds are identified at the ATCC as Dianthus caespitosus NC 1614. This deposit was made in compliance with the Budapest Treaty requirements that the duration of the deposit should be for thirty (30) years from the date of the deposit or for five (5) years after the last request for the deposit at the depository or for the enforceable life of a U.S. patent that matures from this application, whichever is longer. These Dianthus caespitosus seeds will be replenished should it become non-viable at the depository.

All references cited herein are incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. An interspecific Dianthus plant produced by the method comprising the steps of:
a. crossing a Dianthus caespitosus with a Dianthus barbatus;
b. recovering the resulting F₁ hybrid interspecific Dianthus seed; and
c. planting the F₁ hybrid interspecific Dianthus seed and regenerating into plants.

2. The interspecific Dianthus plant of claim 1 wherein said interspecific Dianthus plant exhibits heat tolerance.

3. The interspecific Dianthus plant of claim 1 wherein the plant has a pedigree which includes plant BFP-2081 and derivatives thereof.

4. An interspecific Dianthus plant produced by the method of claim 1.

5. The interspecific Dianthus plant of claim 4 wherein said interspecific Dianthus plant exhibits heat tolerance.

6. Pollen of the plant of claim 4.

7. An ovule of the plant of claim 4.

8. A tissue culture comprising regenerable cells of the plant of claim 4.

9. A cutting of the plant of claim 4.

10. Seed of the plant of claim 4.

11. A heat tolerant interspecific Dianthus plant.

12. A heat tolerant interspecific Dianthus plant wherein said plant has a pedigree which includes plant BFP-2081 and derivatives thereof.

13. Pollen of the plant of claims 11 or 12.

14. An ovule of the plant of claims 11 or 12.

15. A tissue culture comprising regenerable cells of the plant of claims 11 or 12.

16. A cutting of the plant of claims 11 or 12.

17. Seed of the plant of claims 11 or 12.

18. A method of producing an interspecific Dianthus plant having an altered flower color or color pattern, the method comprising the steps of:
a. crossing a Dianthus caespitosus with a Dianthus barbatus;
b. recovering the resulting F₁ hybrid interspecific Dianthus seed;
c. planting the F₁ hybrid interspecific Dianthus seed and regenerating into plants; and
d. treating one of the regenerated plants of step c) or a part of one of the regenerated plants of step c) with electromagnetic radiation or ion beams and selecting a flower from said cultivar having at least one petal exhibiting an altered color or altered color pattern.

19. The method of claim 18 wherein the interspecific Dianthus plant is heat tolerant.

20. The method of claim 18 wherein the plant is irradiated with gamma rays, x-rays or ultraviolet rays.

21. The method of claim 18 wherein the Dianthus plant is irradiated with from about 1.5 to about 3.5 krads of electromagnetic radiation or ion beams.

22. A Dianthus plant comprising a flower having at least one petal which exhibits an altered color or altered color pattern produced by the method of claim 18.

23. Pollen of the plant of claim 22.

24. An ovule of the plant of claim 22.

25. A tissue culture comprising regenerable cells of the plant of claim 22.

26. A cutting of the plant of claim 22.

27. Seed of the plant of claim 22.

28. A method for making heat tolerant interspecific Dianthus plants, the method comprising the steps of:
a. crossing a Dianthus caespitosus with a second Dianthus plant other than Dianthus caespitosus;
b. recovering the resulting F₁ hybrid interspecific Dianthus seed;
c. planting the F₁ hybrid interspecific Dianthus seed and regenerating into plants; and
d. selecting heat tolerant interspecific Dianthus plants.

29. The method of claim 28 wherein the second Dianthus plant is Dianthus barbatus.

30. The method of claim 28 wherein the Dianthus caespitosus is used as the male parent or the female parent.

31. A heat-tolerant interspecific Dianthus plant produced by the method of claim 28.
